(19) United States
(12) Patent Application Publication
(10) Pub. No.: US 2006/0025706 A1
(43) Pub. Date: Feb. 2, 2006

(54) Human body shape management device

(76) Inventor: Yu-Yu Chen, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/902,952

(22) Filed: Aug. 2, 2004

(51) Int. Cl. A61B 5/103; G01B 3/10

(52) U.S. Cl. 600/587; 33/763

(57) ABSTRACT

A human body shape management device mainly includes a digital tape measuring mechanism for taking a user's body measurements and generating a measure signal based on the obtained measurements; a microprocessor for receiving the measure signal and calculating the user's body measurements based on the received measure signal; at least one data storage connected to the microprocessor for storing the user's current body measurements at different areas, and reference and ideal body measurements; a display connected to the microprocessor for displaying the user's current and past body measurements over a long period taken by the digital tape measuring mechanism, and curves indicating changing tendencies of the user's body measurements; and a data communication port connected to the microprocessor for downloading or uploading reference and ideal body measurement data stored in the at least one data storage to a personal or a notebook computer.

### BACKGROUND OF THE INVENTION

### Field of the Invention

The present invention relates to a human body shape management device, and more particularly to a digital management device for human body measurements.

With the prosperous developments in the modern society, people's diets are also diversified. However, improper and surplus diets and lacking in exercise are always important factors resulting in fatness and illness. In assessing a person's health condition, different body measurements are usually an important basis for such assessment.

A cloth measuring tape or a caliper rule is conventionally used to take a person's body measurements, and the measurements so obtained are inaccurate and valueless in terms of managing body measurements.

In other conventional measuring techniques, big-scaled infrared and super-sonic measuring devices have also been used to take body measurements. However, such conventional measuring devices can be used to take only height and measures at some other specific areas, and therefore could not be used in all-round and effective management of a user's body measurements.

Moreover, in the conventional way of body management, the obtained body measurements are manually entered into a computer or recorded in a notebook. This is inconvenient to the user and largely reduces the effect of the body management.

Another problem with the conventional way of taking body measurements is it does not automatically store a large quantity of personal historical data of body measurements, nor does it compare the obtained measurements with reference body measurements or the user's ideal body measurements to effectively achieve the purpose of body management.

### SUMMARY OF THE INVENTION

A primary object of the present invention is to provide a human body shape management device that combines a digital tape measuring mechanism and a numeric display function to effectively manage a user's body measurements.

Another object of the present invention is to provide a human body shape management device for human body measurements that can be carried along with a user to perform the body management at any time and any place, so that the user may accurately control his body conditions all the time.

A further object of the present invention is to provide a human body shape management device for human body measurements that is also capable of data management. In addition to the measuring function, the device of the present invention can also record the measured data and analyze such data on different time basis.

A still further object of the present invention is to provide a human body shape management device for human body measurements that is also capable of storing reference and ideal body measurements at different areas. The user may accurately control his current body conditions from a curve of changing tendency of the body measurements.

A still further object of the present invention is to provide a human body shape management device for human body measurements that integrates a data communication port to download or upload the body measurement data in a data storage of the device to a personal computer or a notebook computer for further data analysis or body pattern simulation, and accordingly effective management of human body measurements.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a schematic view of a human body shape management device according to an embodiment of the present invention;

FIG. 2 shows a first embodiment of a digital tape measuring mechanism included in the present invention;

FIG. 3 is a block diagram showing a control circuit for the present invention;

FIG. 4 shows wave patterns of measure signals generated by magnetic reed switches shown in FIG. 2;

FIG. 5 shows a second embodiment of the digital tape measuring mechanism included in the present invention;

FIG. 6 is a schematic view showing the data and curves of the waist of a user which are measured by the human body shape management device and displayed on the display of the present invention; and

FIG. 7 is a schematic view showing the data and curves of the waist of a user which are measured by the human body shape management device and displayed on the display of the present invention.

### DETAILED DESCRIPTION OF THE PREFERRED EMBODIMENTS

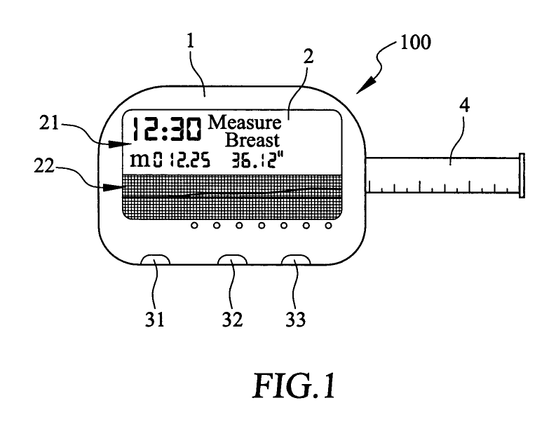

Please refer to FIG. 1 that is a schematic view of a human body shape management device 100 according to an embodiment of the present invention. As shown, the human body shape management device 100 includes a case 1, on which a display 2 is provided for showing a person's current measurement and past measurements over a long period, and curves indicating changing tendencies of the person's measurements at different areas. The display 2 is divided into a numeric and word zone 21 and a graphic zone 22. In the numeric and word zone 21, numerical values and words indicating time, size, etc. are displayed; and in the graphic zone 22, curve patterns related to human body management are shown. The present invention is adapted to measure a user's height, and accurate measurements at different areas. For example, the breast measurement of the user is displayed on the display 2, as shown in FIG. 1.

On the case 1, there are also provided a plurality of keys 31, 32, 33 for operating the human body shape management device 100. The device 100 includes a tape 4 that can be pulled out from one side of the case 1. Like a conventional tape measure, the tape 4 is made of a flexible material. One of the keys, for example key 32, can be preset to be a mode selecting key for selection of the body part to be measured, such as breast, waist or hip. By pressing the mode selecting key and conducting body measurement, measured data are stored to the corresponding reference body measurement data storage of the human body shape management device 100 and used for generating a historical curve showing the past and current data. Accordingly, the user is able to observe the fluctuation of his body measurements at any time.

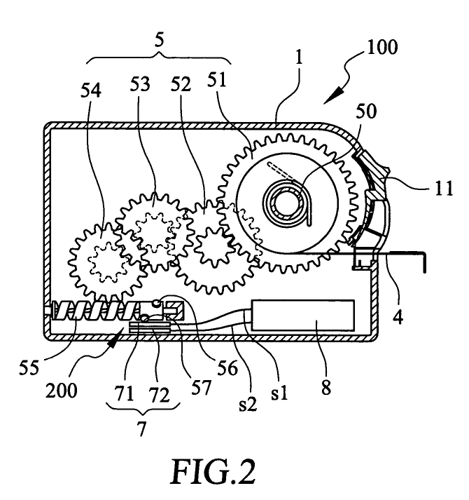

The device 100 of the present invention is internally provided with a digital tape measuring mechanism 200 for measuring a person's height and accurate measurements at different areas in a digitalized manner. FIG. 2 shows a first embodiment of the digital tape measuring mechanism 200, which includes a gear set 5 consisting of gears 51, 52, 53, and 54 provided inside the case 1. The first gear 51 has an axle, around which the tape 4 is wound. A ring-shaped spring 50 is provided on the axle of the first gear 51 for rewinding and keeping the tape 4 in the case 1 when the tape 4 is not pulled outward. A lock 11 is provided on the case 1 near an outlet of the tape 4 for locking the tape 4 in an outward extended position without the risk of being unexpectedly rewound into the case 1.

A screw rod 55 is provided in the case 1 in the vicinity of the last gear 54 of the gear set 5 to mesh with the last gear 54. An end of the screw rod 55 is provided with at least one set of two spaced and staggered magnetic bodies 56, 57. At least one set of two sensing elements are provided in the vicinity of the two magnetic bodies 56, 57 for sensing the state of the magnetic bodies 56, 57. The sensing elements may be, for example, two magnetic reed switches 71, 72 electrically connected to a circuit board 8 via conductors.

The gears 51, 52, 53, and 54 possessing particular gear ratios are able to magnify the scale measured by the digital tape measuring mechanism 200. When the tape 4 is pulled out for measurement, the gears 51, 52, 53, and 54 rotate in series, and finally the gear 54 drives the screw rod 55 to turn in a magnified manner. Thereby, the precision of the measured data is enhanced. Supposing the gear ratio of the gears 51, 52, 53, and 54 to be 10, when the tape 4 is pulled out for 1 cm, the screw rod 55 is driven to rotate for a distance of 10 cm, and the magnetic reed switches 71, 72 of the digital tape measuring mechanism 200 are actuated to generate a signal representing 10 cm. Thereby, the precision of the digital tape measuring mechanism 200 is increased for ten times due to the magnification of the magnitude.

The gear set 5, the screw rod 55, the magnetic bodies 56, 57, and the magnetic reed switches 71, 72 together constitute the digital tape measuring mechanism 200 in the present invention. When a user pulls the tape 4 outward, all the gears 51, 52, 53, 54 of the gear set 5 are caused to rotate synchronously and bring the screw rod 55 to rotate. Whenever the magnetic bodies 56, 57 on one end of the screw rod 55 are rotated by one turn, the magnetic reed switches 71, 72 are actuated to generate a set of two measure signals s1, s2, which are sent to a control circuit 80 on the circuit board 8.

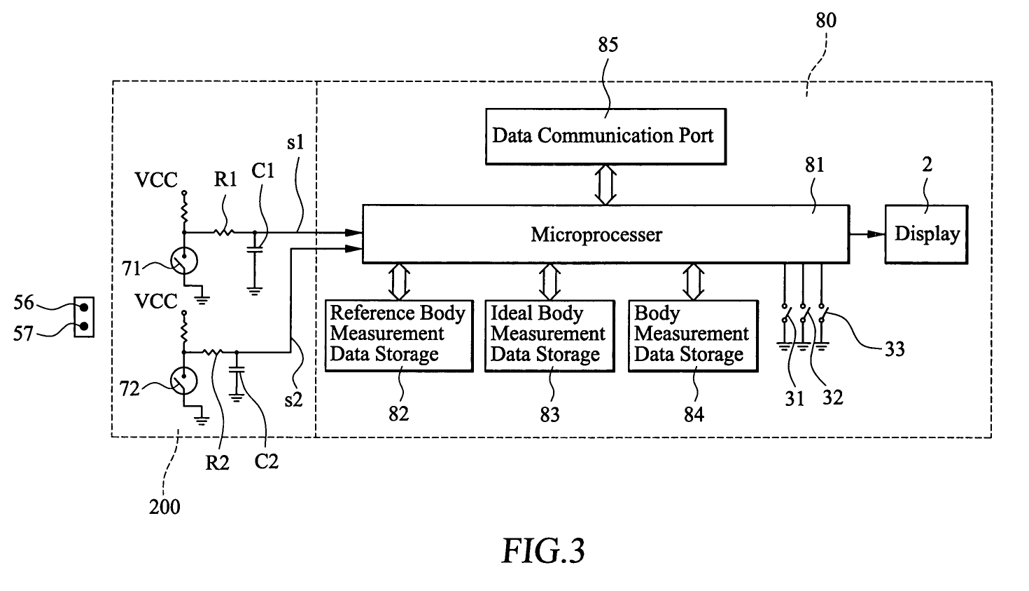

FIG. 3 is a block diagram showing the control circuit 80 of the human body shape management device 100. The control circuit 80 mainly includes a microprocessor 81 for calculating, storing, and displaying different measuring values taken at different areas of a user. The microprocessor 81 is electrically connected to the display 2 and the keys 31, 32, 33. The measure signals s1, s2 generated by the magnetic reed switches 71, 72 in response to the rotation of the screw rod 55 are sent to the control circuit 80 on the circuit board 8.

Before being sent to the microprocessor 81, the measure signal s1 generated by the magnetic reed switch 71 passes a de-bouncing circuit consisting of a resistance R1 and a capacitor C1 to remove any noise from the signal s1. Similarly, before being sent to the microprocessor 81, the measure signal s2 generated by the magnetic reed switch 72 passes a de-bouncing circuit consisting of a resistance R2 and a capacitor C2 to remove any noise from the signal s2.

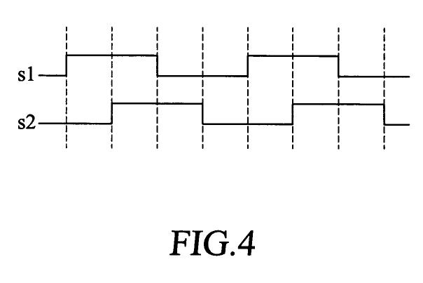

Whenever the magnetic reed switches 71, 72 generate one set of two measure signals s1, s2, the measure signals s1, s2 are immediately sent to the microprocessor 81 in the control circuit 80. The measure signals s1, s2 generated by the magnetic reed switches 71, 72 have waveforms with phase difference, as shown in FIG. 4.

With the signal sensing function of the magnetic reed switches 71, 72, it is possible to calculate and find whether the screw rod is rotating forward or reversely, and derive an accurate measured length or size. Then, the microprocessor 81 is able to calculate data about the user's body measurements, such as bust, waist, height, etc. based on the received measure signals s1, s2.

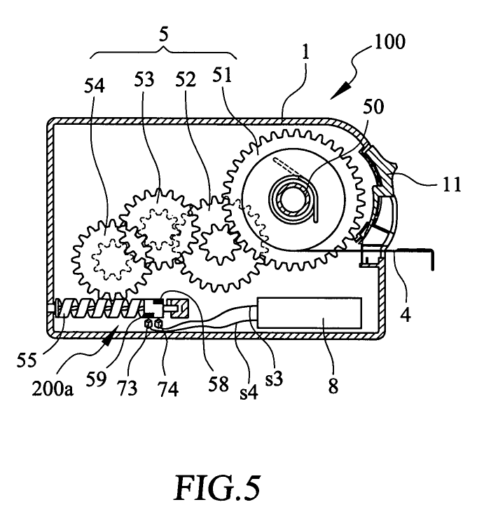

FIG. 5 shows a second embodiment of the digital tape measuring mechanism 200a. The digital tape measuring mechanism 200a is generally similar to the digital tape measuring mechanism 200, except that the screw rod 55 is provided at one end with a pair of reflective sensing zones 58, 59, which may be, for example, bar codes printed or pasted on the end of the screw rod; and a pair of reflective opto-sensors 73, 74 are correspondingly provided in the vicinity of the reflective sensing zones 58, 59 to sense the number of revolutions of the screw rod 55. The reflective opto-sensors 73, 74 also generate measure signals s3, s4 in response to the sensing thereof. And, signals s3, s4 also have waveforms with phase difference as shown in FIG. 4.

It is also possible for the microprocessor 81 to connect to a reference body measurement data storage 82, in which numerical values representing reference body measurements at different areas are stored, and to an ideal body measurement data storage 83, in which ideal values for the user's body measurements at different areas are stored. A body measurement data storage 84 is coupled with the microprocessor 81, which stores the measurements taken from the user by the digital tape measuring mechanism 200. After the digital tape measuring mechanism 200 takes the measurement from the user, the measurement is transmitted to and automatically stored in the body measurement data storage 84.

The microprocessor 81 may also be connected to at least one data communication port 85, so that the reference body measurement data stored in the reference body measurement data storage 82 and the ideal body measurement data stored in the ideal body measurement data storage 83 may be downloaded or uploaded to a personal computer or a notebook computer for further data analysis or body pattern simulation, and accordingly effective management of human body measurements.

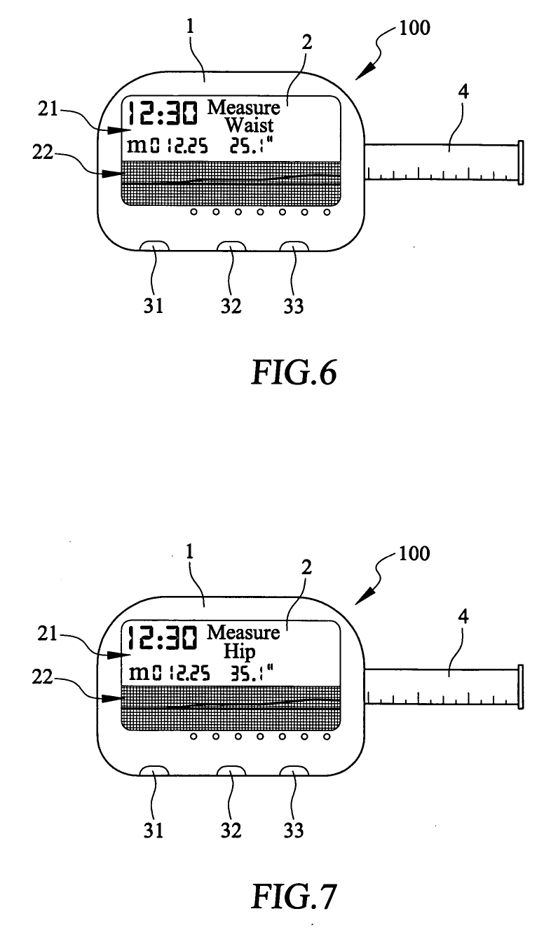

The human body shape management device of the present invention can be used for measuring the user's height and accurate measurements at different areas. For example, the human body shape management device can be used to measure breast measurement as shown in FIG. 1, waist measurement as shown in FIG. 6 or hip measurement as shown in FIG. 7. The human body shape management device 100 can also be used to take measurements of the arm, shoulder, thigh and any parts of the user. The measurements taken are transmitted to and automatically stored in the body measurement data storage 84. By pressing the keys 31, 32, 33, the measurement of particular part of the user at specific time, such as the breast measurement of last month, is retrieved from the body measurement data storage 84 and displayed on the display 2.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A human body shape management device, comprising:
a digital tape measuring mechanism for taking a user's body measurements at different areas, and generating a measure signal based on the obtained measurements;
a microprocessor for receiving said measure signal generated by said digital tape measuring mechanism and calculating the user's body measurements at different areas based on the received measure signal;
at least one data storage connected to said microprocessor for storing said user's body measurements at different areas taken by said digital tape measuring mechanism; and
a display connected to said microprocessor for displaying said user's body measurements at different areas taken by said digital tape measuring mechanism and the user's past body measurements taken and recorded over a long period, and curves indicating changing tendencies of the user's body measurements at different areas.

2. The human body shape management device as claimed in claim 1, further comprising a data communication port connected to said microprocessor for downloading or uploading reference body measurement data and ideal body measurement data stored in said at least one data storage to a personal computer or a notebook computer.

3. The human body shape management device as claimed in claim 1, wherein said data storage comprises a reference body measurement data storage connected to said microprocessor for storing numerical values representing reference body measurements at different areas, and an ideal body measurement data storage connected to said microprocessor for storing ideal values for the user's body measurements at different areas.

4. The human body shape management device as claimed in claim 1, further comprising a body measurement data storage for storage of measurements taken from the user, in which after the human body shape management device takes the measurement from the user, the measurement is transmitted to and automatically stored in the body measurement data storage

5. The human body shape management device as claimed in claim 1, wherein said digital tape measuring mechanism includes:
an outward pullable tape;
a gear set adapted to rewind said tape and rotate in response to a length of said tape that is pulled outward;
a screw rod meshed with said gear set and thereby driven by said gear set; and
at least one sensing element for sensing a rotating state of said screw rod and generating said measure signal to said microprocessor.

6. The human body shape management device as claimed in claim 5, wherein said sensing element is a magnetic reed switch, and wherein said screw rod is provided at an end with at least one set of magnetic bodies corresponding to said at least one sensing element.

7. The human body shape management device as claimed in claim 5, wherein the gear set has a predetermined gear ratio, so that when the pullable tape is pulled out for measurement, the gear drives the screw rod to turn in a magnified rotation manner and thereby the sensing element senses a magnified scale in correspondance to the magnified rotation of the screw rod.
